(19) United States
(12) Patent Application Publication
(10) Pub. No.: US 2021/0278359 A1
(43) Pub. Date: Sep. 9, 2021

(54) ENVIRONMENTAL SENSING DEVICE AND APPLICATION METHOD THEREOF

(71) Applicant: Soiltech Wireless Inc, Dover, DE (US)

(72) Inventor: Ehsan Soltanmohammadi, Swansea (GB)

(21) Appl. No.: 16/809,592

(22) Filed: Mar. 5, 2020

(51) Int. Cl. G01N 27/22; G01K 13/00; G01K 15/00; G01P 15/18; G01S 19/01

(52) U.S. Cl. CPC G01N 27/223; G01K 13/00; A01C 7/00; G01P 15/18; G01S 19/01; G01K 15/005

(57) ABSTRACT

The present invention provides an environmental sensing device comprising: a body having an accommodating space inside; at least one first conducting unit disposed outside the body; at least one second conducting unit disposed outside the body and corresponding to the first conducting unit; an electric measuring unit disposed in the accommodating space for measuring current, voltage, capacitance and temperature of the surrounding environment corresponding to the first conducting unit and the second conducting unit; wherein the electric measuring unit further transmits a first signal according to the measurement of the current, voltage, capacitance and temperature of the environment; a processing unit disposed in the accommodating space for receiving the first signal and calculating moisture of the surrounding environment according to an environmental data and the measurements of the current, voltage, capacitance and temperature of the surrounding environment; and a power supply unit disposed in the accommodating space for supplying electric power to the electric measuring unit and the processing unit. The environmental sensing device is able to provide continuously monitor the environmental conditions of the crops of different stages of planting, transport and storage, etc.

### BACKGROUND OF THE INVENTION

### 1. Field of the Invention

The present disclosure generally relates to an environmental sensing device and application method thereof, and, more particularly, to an environmental sensing device for sensing the environmental conditions of crops and transmitting the environmental conditions wirelessly to allow users to monitor the environmental conditions and take action based on the data collected.

### 2. Description of the Related Art

In order to achieve automated management and to ensure the quality of crops, it is necessary to monitor the environmental conditions of the crops during the growth, transport and storage of the crops. Further, the environmental conditions may include temperature, humidity and moisture, etc. Taking the potato crop as an example, it is necessary to monitor the temperature, humidity and moisture of the soil during the growth of the potato. Monitoring such environmental conditions and collect relative data are necessary for automated management. Also, such monitoring process is helpful to properly manage moisture of the planting soil, to avoid crop diseases, so as to ensure the quality and the yield of the crop. Additionally, by continuously monitoring the environmental conditions during other stages such as harvesting, transport or storage, etc., of the crops, to prevent the crops from being susceptible to disease or spoilage.

Conventional moisture sensing devices perform measurements according to osmotic balance. However, conventional moisture sensing devices are used without any self-contained waterproof protective case, so the measurement accuracy is easily affected.

Conventional moisture sensing devices usually include a processor and at least one probe electrically connected to the processor by a wire or a cable. The probe of a conventional moisture sensing device is inserted into soil and the antenna/processor part of the wire would be exposed to air for farmers to read the measurement. Consequently, such setup may be interfered by other farming equipments or processes. Such moisture sensing device also needs to be removed from the field before harvesting, otherwise conventional moisture sensing device may interfere, or can be damaged by, the harvesting process. Hence, the environmental conditions may not be fully or continuously monitored during the harvesting, transportation or storage stage.

Therefore, how to monitor the environmental conditions (e.g. soil moisture and/or humidity, etc.) continuously with reliability and accuracy during different stages (e.g. planting, growth, harvesting, storage or transportation, etc.) of the crop has become an urgent problem to be solved in the industry.

### SUMMARY OF THE INVENTION

In light of solving the foregoing problems of the prior art, the present invention provides an environmental sensing device comprising a body, at least one first conducting unit, at least one second conducting unit, an electric measuring unit, a processing unit and a power supply unit.

The body has an accommodating space inside. The at least one first conducting unit is disposed outside of the body. The at least one second conducting unit is disposed outside the body and corresponding to the first conducting unit. The electric measuring unit is disposed in the accommodating space for measuring current, voltage, capacitance and temperature of the surrounding environment corresponding to the first conducting unit and the second conducting unit; wherein the electric measuring unit further transmits a first signal according to the measurement of the current, voltage, capacitance and temperature of the environment. The processing unit is disposed in the accommodating space for receiving the first signal and calculating a moisture of the surrounding environment according to an environmental data and the measurement of the current, voltage, capacitance and temperature of the surrounding environment. The power supply unit is disposed in the accommodating space for supplying power to the electric measuring unit and the processing unit.

In an embodiment, the environmental sensing device according to the present invention further comprises a temperature sensing unit disposed in the accommodating space for sensing temperature of the surrounding environment and transmitting a second signal, wherein the processing unit is further used to receive the second signal and to calibrate the measurement of the temperature of the surrounding environment by the second signal. The processing unit further calculates the moisture of the surrounding environment according to the environmental data, the measurement of the current, voltage and capacitance of the surrounding environment and the calibrated temperature of the surrounding environment.

In an embodiment, the environmental sensing device according to the present invention further comprises a communication unit disposed in the accommodating space and electrically connected to the processing unit for communicating with an external data processing device, such as a cellphone or personal computer.

In an embodiment, the environmental sensing device according to the present invention further comprises a positioning unit disposed in the accommodating space and electrically connected to the processing unit for tracking the location of the environment sensing device.

In an embodiment, the environmental sensing device according to the present invention further comprises a temperature sensing unit. The temperature sensing unit is disposed in the accommodating space for sensing temperature of the surrounding environment and transmitting a second signal; the communication unit transmits a third signal according to the location of the environment sensing device to an database, wherein the database pre-saves worldwide geographic information; the database sends the geographic information of the location of the environment sensing device according to the third signal to the communication unit, and the processing unit is further used to receive the second signal and calibrating the measurement of the temperature of the surround environment by the second signal; wherein the processing unit further calculates the moisture of the surrounding environment according to the environmental data, the measurement of the current, voltage and capacitance of the surrounding environment, the calibrated temperature of the surrounding environment and the geographic information.

In an embodiment, the environmental sensing device according to the present invention further comprises a storage unit disposed in the accommodating space for receiving and recording the measurement of the current, voltage, capacitance and temperature of the surrounding environment, the moisture calculated by the processing unit and the corresponding time.

In an embodiment, the environmental sensing device according to the present invention further comprises an acceleration sensing unit disposed in the accommodating space and electrically connected to the processing unit for sensing an external force received by the body.

In an embodiment, the environmental sensing device according to the present invention further comprises a fixing frame disposed in the accommodating space, wherein the fixing frame has at least one cavity for fixing components disposed in the accommodating space.

In an embodiment, the first conducting unit and the second conducting unit are annular and disposed around the outside of the body.

In an embodiment, the first conducting unit and the second conducting unit are made of stainless steel.

The present invention further provides an application method of the environmental sensing device, comprising the steps of burying the environmental sensing device in the soil in which crops grow when planting the crops; harvesting the environmental sensing device together with the crop when harvesting the crops; transporting the environmental sensing device together with the crop when transporting the crops; and storing the environmental sensing device together with the crop when the crops are stored.

The present invention further provides a method for measuring environmental condition around crops, comprising: burying the environmental sensing device in the soil where the crops grow when planting the crops.

The environmental sensing device comprises a body, at least one first conducting unit, at least one second conducting unit, an electric measuring unit, a communication unit, a positioning unit, a temperature sensing unit, a processing unit.

The body has an accommodating space inside. The electric measuring unit, the communication unit, the positioning unit, the temperature sensing unit and the processing unit are disposed in the accommodating space.

The at least one first conducting unit and the at least one second conducting unit are disposed outside the body and the at least one second conducting unit is corresponding to the first conducting unit; wherein the at least one first conducting unit and the at least one second conducting unit touch the soil where the crops grow.

The electric measuring unit is for measuring current, voltage, capacitance and temperature of the surrounding environment corresponding to the first conducting unit and the second conducting unit; and wherein the electric measuring unit further transmits a first signal according to the measurement of the current, voltage, capacitance and temperature of the environment.

The communication unit is electrically connected to the processing unit for communicating with an external data processing device.

The positioning unit is electrically connected to the processing unit for tracking a location of the environment sensing device.

The temperature sensing unit is for sensing temperature of the surrounding environment and transmitting a second signal.

The communication unit transmits a third signal according to the location of the environment sensing device to a database, wherein the database pre-saves worldwide geographic information and the database sends the geographic information of the location of the environment sensing device to the communication unit.

The processing unit is for receiving the first signal and the second signal and calibrating the measurement of the temperature of the surrounding environment by the second signal. The processing unit further calculates soil moisture of the soil where the crops grow according to the environmental data, the measurement of the current, voltage and capacitance of the surrounding environment, the calibrated temperature of the surrounding environment and the geographic information.

In an embodiment, the method for measuring environmental condition around crops further comprises: harvesting the environmental sensing device together with the crop when harvesting the crops.

In this embodiment, the method for measuring environmental condition around crops further comprises: disposing an acceleration sensing unit in the accommodating space and electrically connecting the acceleration sensing unit to the processing unit for sensing an external force received by the body and measuring g-forces incurred during harvest and transportation by the acceleration sensing unit; and transmitting the external force and the g-force to an external data processing device by the communication unit.

In an embodiment, the method for measuring environmental condition around crops further comprises: transporting the environmental sensing device together with the crop when transporting the crops.

In this embodiment, the method for measuring environmental condition around crops further comprises: disposing a humidity sensing unit in the accommodating space for sensing humidity of the surrounding environment of the crops; tracking a location of the environment sensing device and the crops by the positioning unit; sensing temperature of the surrounding environment of the crops by the temperature sensing unit; sensing an external force received by the body and measuring g-forces incurred during transportation by the acceleration sensing unit; and transmitting the humidity, the location, the temperature and the external force to an external data processing device by the communication unit to allow users to monitor the environmental conditions while in transit.

In an embodiment, the method for measuring environmental condition around crops further comprises: storing the environmental sensing device together with the crop when the crops are stored.

In this embodiment, the method for measuring environmental condition around crops further comprises: sensing humidity of the surrounding environment of the crops by the humidity sensing unit; tracking a location of the environment sensing device and the crops by the positioning unit; sensing temperature of the surrounding environment of the crops by the temperature sensing unit; and transmitting the humidity, the location and the temperature to an external data processing device by the communication unit to allow users to monitor the environmental conditions while in storage.

The environmental sensing device according to the present invention comprises at least one first conducting unit and at least one second conducting unit disposed outside the body. The environmental sensing device senses the current, voltage, capacitance and temperature corresponding to the first conducting unit and the second conducting unit by the electric measuring unit, and a moisture of the surrounding environment is further calculated by the processing unit. The moisture provided by the environmental sensing device according to the present invention is more accurate because it is calculated according to the values of current, voltage, capacitance and temperature combined with other environmental data. Besides, the environmental sensing device according to the present invention is able to monitor the whole process continuously from planting to storage to transport.

### BRIEF DESCRIPTION OF THE DRAWINGS

FIG. 1 illustrates a schematic view of the environmental sensing device according to the first embodiment of the present invention.

FIG. 2 illustrates a functional block diagram of internal components of the environment sensing device according to the first embodiment of the present invention.

FIG. 3 illustrates a functional block diagram of internal components of the environment sensing device according to the second embodiment of the present invention.

FIG. 4 illustrates a cross-sectional view of the environment sensing device according to a third embodiment of the present invention.

FIG. 5 illustrates a schematic structural view of the first conducting unit and the second conducting unit according to a fourth embodiment of the present invention.

FIG. 6 illustrates a flow chart showing steps of the application method of the environment sensing device according to the present invention.

FIG. 7 illustrates a flow chart showing steps of the method for measuring environmental condition around crops according to the present invention.

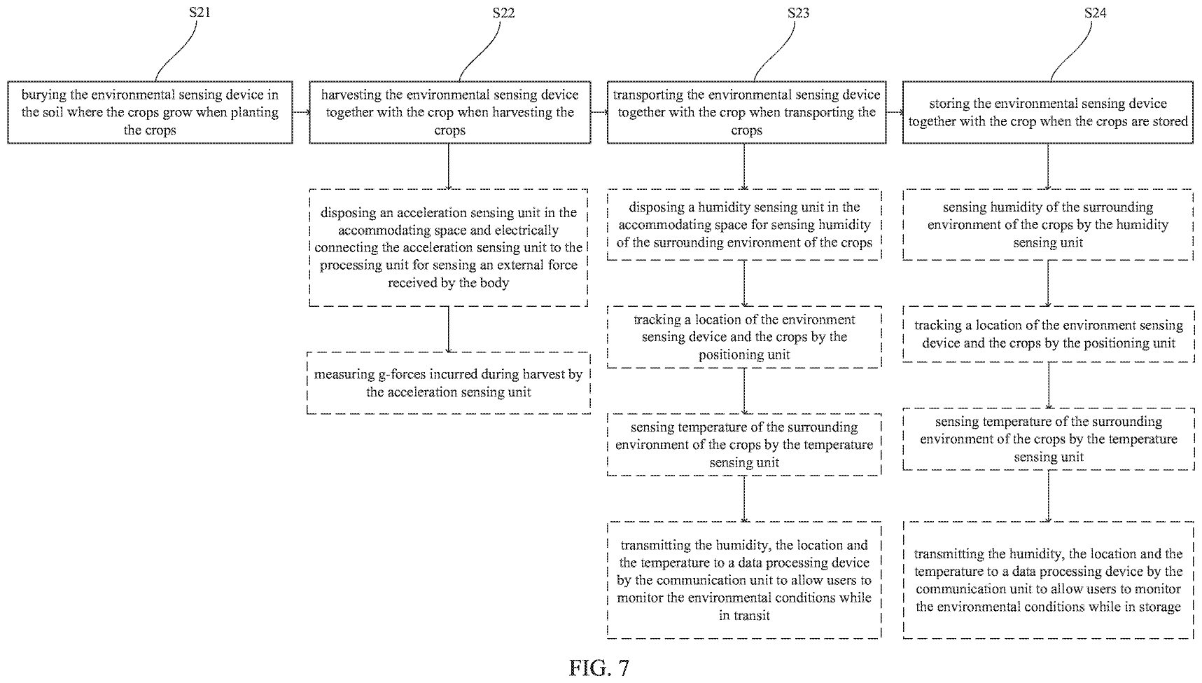

### DETAILED DESCRIPTION

The present invention is described by the following specific embodiments. Those with ordinary skills in the arts can readily understand other advantages and functions of the present invention after reading the disclosure of this specification. Any changes or adjustments made to their relative relationships, without modifying the substantial technical contents, are also to be construed as within the range implementable by the present invention.

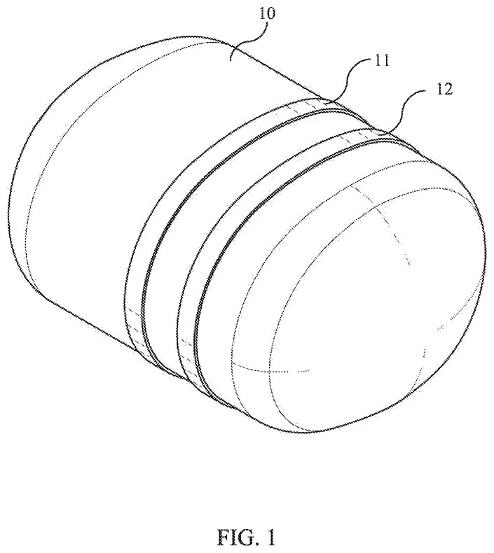

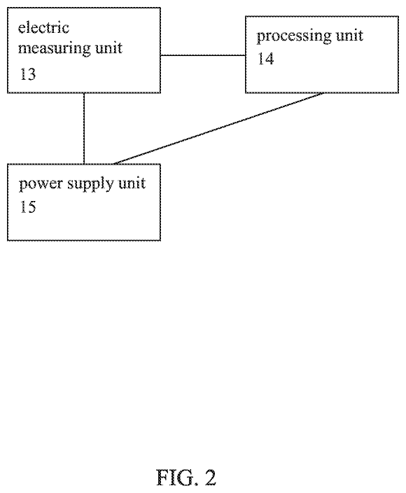

Please refer to FIG. 1 and FIG. 2. FIG. 1 illustrates a schematic view of an environmental sensing device according to a first embodiment of the present invention, and FIG. 2 illustrates a functional block diagram of internal components of the environment sensing device according to the first embodiment of the present invention. As shown in FIGS. 1 and 2, the environmental sensing device of the present invention may comprise a body 10, at least one first conducting unit 11, at least one second conducting unit 12, an electric measuring unit 13, a processing unit 14 and a power supply unit 15.

In an embodiment, the body 10 may be waterproof and crash-resistant. The body 10 may be made of waterproof and impact resistant materials. The body 10 has an accommodating space inside for placing various electronic components. Since the shape of the body 10 may be intact and the body 10 may be waterproof and crash-resistant, the electronic components inside the body 10 may be fully covered, and cannot be easily damaged. Therefore, the environment sensing device according to the present invention may measure environmental conditions with a better reliability. In a further embodiment, the dimension, shape, or contour of the body 10 can be designed to be comparable to that of the desired crop.

In an embodiment, the at least one first conducting unit 11 and the at least one second conducting unit 12 may be disposed outside the body 10. The second conducting unit 12 may correspond to the first conducting unit 11. In particular, the first conducting unit 11 and the second conducting unit 12 may have the same shape and length and have a substantially equidistant gap between. When the environment sensing device according to the present invention is placed near the crops, the at least one first conducting unit 11 and the at least one second conducting unit 12 may work similar to capacitive elements, and a substance between them (such as soil, water or air, etc.) may act as the dielectric in between. Subsequently, the capacitance value of the conducting units 11 and 12 are affected by the ambient moisture level, which is affected by factors such as temperature. Therefore, the at least one first conducting unit 11 and the at least one second conducting unit 12 may sense the current, voltage, capacitance and temperature, etc., of the surrounding environment at the same time. Since the at least one first conducting unit and the at least second conducting unit may be made of metal, they may provide very fast electrical factors and temperature sensing. Furthermore, since the at least one first conducting unit 11 and the at least second conducting unit 12 are disposed outside of the body 10, the effectiveness of the waterproofing of the body 10 can also be measured.

In an embodiment, the electric measuring unit 13 may be disposed in the accommodating space. The electric measuring unit 13 may measure the current, voltage, capacitance and temperature, etc., of the surrounding environment corresponding to the first conducting unit 11 and the second conducting unit 12. The electric measuring unit 13 can be electrically connected to the first conducting unit 11 and the second conducting unit 12. The electric measuring unit 13 may further transmit a first signal according to the obtained measurement of the current, voltage, capacitance and/or temperature, etc., of the environment to the processing unit 14.

In an embodiment, the processing unit 14 may be disposed in the accommodating space for receiving the first signal. The processing unit 14 may calculate moisture of the surrounding environment according to environmental data and the measurements of current, voltage, capacitance and temperature. According to the present invention, the environmental data can include the dielectric constant of soil or air at different moisture levels. In other words, the environment sensing device according to the present invention can measure the moisture of the surrounding environment in real time. This can help manage the quality of the crops and also automate the process of doing so. For instance, the electric measuring unit 13 and the processing unit 14 may comprise, but not limited to, an IC, a PLC or the like.

In an embodiment, the power supply unit 15 may be disposed in the accommodating space for supplying electric power to the electric measuring unit 13 and the processing unit 14. The power supply unit 15 can be, for example, a battery or any suitable power supply.

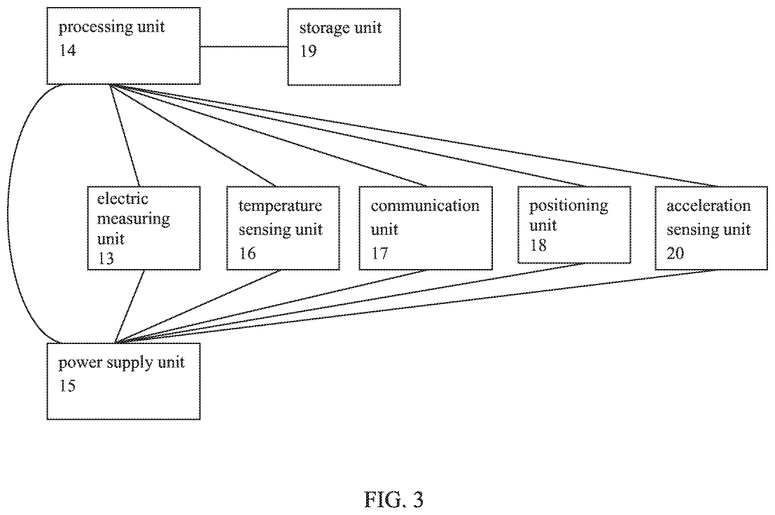

Please refer to FIG. 3 which illustrates a functional block diagram of internal components of the environment sensing device according to a second embodiment of the present invention. In this embodiment, the environmental sensing device according to the present invention may further comprise a temperature sensing unit 16 disposed in the accommodating space for sensing temperature of the surrounding environment. The temperature sensing unit 16 may also transmit a second signal containing temperature information. Preferably, the processing unit 14 may be further used to receive the second signal and calibrate the measurement of the temperature of the surround environment according to the second signal, so as to obtain a more accurate temperature reading. The processing unit 14 may further calculate the moisture of the surrounding environment according to the environmental data, the measurement of the current, voltage and capacitance of the surrounding environment and/or the calibrated temperature of the surrounding environment, etc. The capacitance is temperature dependent and moisture is affected by the temperature. The environmental sensing device according to the present invention may provide a more accurate and precise temperature reading of the surrounding environment by the calibration of the temperature sensing unit 16, resulting in a more reliable calculation of moisture.

In an embodiment, the environmental sensing device according to the present invention may further comprise a communication unit 17 disposed in the accommodating space and electrically connected to the processing unit 14 for wirelessly communicating with an external data processing device. The communication unit 17 may be a wireless communication component using LTE technology (CAT-M1, NB-IOT), but it is not limited thereto. The environment sensing device of the present invention can exchange information with an external data processing device (such as a mobile device, a computer, a server, etc.) through the communication unit 17. The environment sensing device may transmit real-time information such as moisture levels and temperature to the external data processing device or receive environmental data from the external data processing device wirelessly.

In an embodiment, the environmental sensing device according to the present invention further comprises a positioning unit 18 disposed in the accommodating space and electrically connected to the processing unit 14 for tracking the location of the environment sensing device. For example, the positioning unit 18 may be an electronic component such as a global positioning system (GPS) module, but it is not limited thereto. The environment sensing device according to the present invention may track the location of the environment sensing device by the positioning unit 18. The location of the environment sensing device can be useful in identifying conditions of the crops, such as whether the crops are currently at the stage of growing, transportation or storage, etc.

Preferably, the communication unit may transmit a third signal according to the location of the environment sensing device to an external database, wherein the database pre-saves worldwide geographic information or meteorological information. The database may send the geographic information or meteorological information of the location of the environmental sensing device to the communication unit according to the third signal. The processing unit 14 may further calculate the moisture of the surrounding environment according to the environmental data, such as the measurement of the current, voltage and capacitance of the surrounding environment and the calibrated temperature of the surrounding environment and the geographic information or meteorological information, etc.

Since soil moisture reacts differently in different soil types, the environmental sensing device according to the present invention can be pre-calibrated so as to correspond to different soil types, such as sand, silt, clay, etc. The positioning unit 18 may be used to determine the type of soil in which the environmental sensing device is disposed, by communicating with the database and the environmental sensing device can then automatically switch the soil moisture profile to match that of the soil in which the environmental sensing device is disposed, so as to provide a more accurate moisture reading.

In an embodiment, the environmental sensing device according to the present invention may further comprise a storage unit 19 disposed in the accommodating space for receiving and recording the measurement of the current, voltage, capacitance and temperature of the surrounding environment, the data of the moisture calculated by the processing unit 14, and the relative event/time log, etc. For example, the storage unit 19 can be a memory, but it is not limited thereto. Environmental data such as changes of moisture through a predetermined period of time, can be stored and accessed in the storage unit 19. Further, the storage unit 19 may be used as a back-up memory to save the measurements and/or the calculated values by the environmental sensing device according to the present invention. In an embodiment, the environmental sensing device according to the present invention may further comprise an acceleration sensing unit 20 disposed in the accommodating space and electrically connected to the processing unit for sensing an external force received by the body 10. For example, the acceleration sensing unit 20 can be a 3-axis accelerometer, but it is not limited thereto. The acceleration sensing unit 20 can sense the external force received by the crops during different stages such as harvesting and transportation. According to the present invention, the external forces may be incurred by other objects hitting the environmental sensing device or otherwise by the environmental sensing device hitting something else. Crops may be harvested using heavy machinery and pass through multiple pieces of machineries and locations, and as such there is a high potential for them to be dropped from excessive heights or at excessive speeds which can cause bruising to the crops. Bruising may reduce the quality and quantity of the crops and may further in turn create disease. This process can be monitored by the environmental sensing device according to the present invention, which may advise the farmers on how to better manage their harvesting and crop transportation operations. According to the present invention, the environmental sensing device may be rugged, waterproof, rough and so on to allow the users to harvest the crops without having to remove the environmental sensing device from the field.

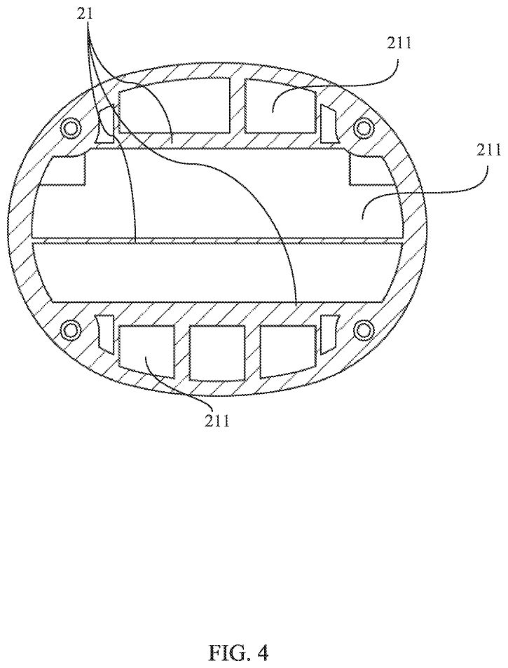

Please refer to FIG. 4. FIG. 4 illustrates a cross-sectional view of the environment sensing device according to a third embodiment of the present invention. In an embodiment, the environmental sensing device according to the present invention may further comprise a fixing frame 21 disposed in the accommodating space for securing components disposed in the accommodating space. The fixing frame 21 may divide the accommodating space into a plurality of small spaces, which means the fixing frame may have at least one cavity 211 illustrated in FIG. 4 and some of which are denoted as 211. The components of the environment sensing device according to the present invention may be fixed in the cavities correspondingly. In an embodiment of the present invention, the environmental sensing device may comprise two antennas which may slide into their own independent cavities. These two cavities for the antennas may be designed to be as far away from battery as possible so as to avoid interference, in their own cavities to protect them and located there because that place may promote the most optimal signal strength. Therefore, moving or shaking the environmental sensing device would not cause damage to the internal components or reduce the efficacy of the environmental sensing device. In other words, the fixing frame 21 may protect the components from undesired conditions such as vibrations and drops that the environmental sensing device may incur to ensure continuous operation of the environmental sensing device. By doing so, the fixing frame 21 may increase reliability of the environment sensing device according to the present invention.

According to the present invention, the body 10 of the environmental sensing device may further comprise a plastic cover for the components to act as a shield during any drops.

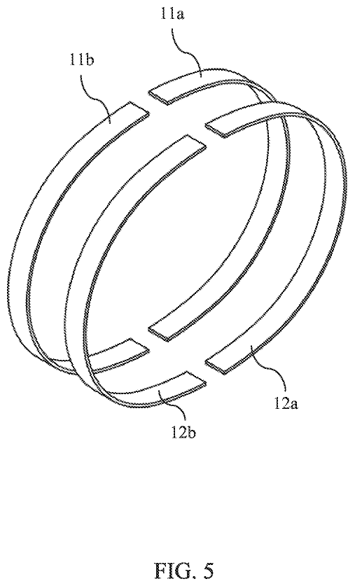

Please refer to FIG. 5 which illustrates a schematic structural view of the first conducting unit and the second conducting unit according to a fourth embodiment of the present invention. In an embodiment, the first conducting units 11a, 11b and the second conducting units 12a, 12b may be designed in a shape of an arc or an arch in accordance with the shape of the body 10. The first conducting units 11a, 11b and the second conducting units 12a, 12b may be disposed around the outside of the body 10. It should be noted that, in the embodiment of FIG. 5, there are a set of two first conducting units 11a, 11b and a set of two second conducting units 12a, 12b. In this embodiment, the electric measuring unit 13 can sense measurements (including the current, the voltage, the capacitance and the temperature) of the two sets of the first conducting units and the second conducting units. By these two sets of measurements, two measures of environmental moisture can be calculated by the environmental sensing device of the present invention; one is the environmental moisture corresponding to the positions of the first conducting unit 11a and the second conducting unit 12a, and the other is the environmental moisture corresponding to the positions of the first conducting unit 11b and the second conducting unit 12b. The number of the first conducting unit 11 and the second conducting unit 12 can be increased or decreased in sets as needed to sense the environmental moisture, as more moisture readings may provide a more accurate measurement of the environmental conditions.

In another embodiment, the environmental sensing device according to the present invention may comprise at least two first conducting units, at least two second conducting units and at least two electric measuring units. Each of the electric measuring units may measure the current, the voltage, the capacitance and the temperature, etc., of each set of the first conducting units and the second conducting units.

In another embodiment, the environmental sensing device according to the present invention may comprise two or more temperature sensing units.

Regarding the abovementioned embodiments, the at least two conducting units and electric measuring units and the at least two temperature sensing units may be functionally independent (i.e. the conducting units, the electric measuring units and the temperature sensing units have no overlapping functions to each other) to better improve the accuracy and to prevent any data inconsistencies. The conducting units, the electric measuring units and the temperature sensing units are also omitted from accessing history data to prevent any discrepancies which might influence the correctness of data.

According to another embodiment of the present invention, the environmental sensing device may further comprise a common moisture sensor which is functionally independent of the environmental sensing device. The common moisture sensor may be any moisture sensors known in the art. In this embodiment, the common moisture sensor may sense the moisture, which may be compared with the moisture calculated by the processing unit 14 for the users' reference.

In an embodiment, the first conducting unit 11 and the second conducting unit 12 may be made of conduction materials which are resistant to be oxidized, such as stainless steel or the like, but they are not limited thereto. Since easily oxidized materials such as copper or copper plating material may degrade device performance or contaminate the crop and the soil. In a preferred embodiment, the first conducting unit 11 and the second conducting unit 12 may be made of stainless steel. It also ensures a longer life of the environmental sensing device of the present invention.

According to the present invention, the environmental sensing device may further conduct the calculation with a noise filtering algorithm. For power consumption reasons, signal emitted by the first conducting unit and the second conducting unit may be low powered. In order to reduce the impact of background noise and unpredictable environmental surroundings, several techniques may be used in the present invention.

For example, to eliminate white noise type interference like thermal noise, the same signal may be sent multiple times and a low pass filter may be applied in. Environmental interferences are more unpredictable, and due to an object may responses differently on different frequency, multiple different frequencies are used and the results are compared to get a coherent trend. After the two stages, the number is kept in a storage buffer. Then filters, such as Kalman filter or the like, may be used to deprive the longer trend.

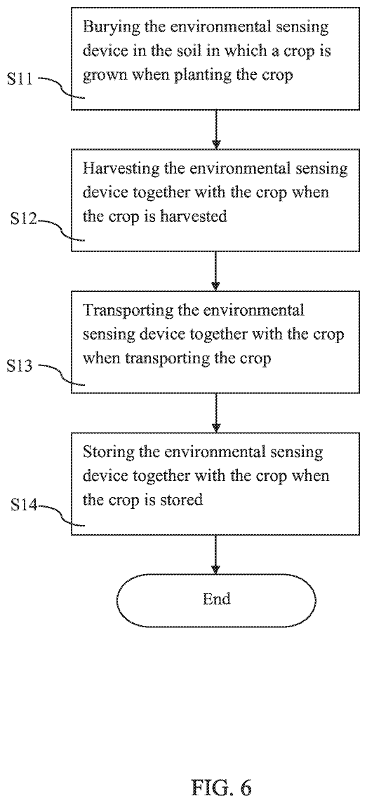

Please refer to FIG. 6 which illustrates a flow chart illustrating an application method of the environment sensing device according to a fifth embodiment of the present invention. Steps of the application method of the environmental sensing device include:

S11. Burying the environmental sensing device in the soil in which crops grow when planting the crops;

S12. Harvesting the environmental sensing device together with the crops when harvesting the crops;

S13. Transporting the environmental sensing device together with the crops when transporting the crops; and

S14. Storing the environmental sensing device together with the crops when the crops are stored.

In the application method according to the present invention, the aforementioned environmental sensing device is disposed near the crops. In an embodiment, the dimension and shape of the environmental sensing device can be designed with reference to the crop (such as a potato) to be monitored. By doing so, the moisture and other environmental conditions sensed by the environmental sensing device are substantially the same as the actual environmental conditions around the crops.

In step S11, the environment sensing device may be buried in the soil close to the target crops, e.g. close to the root of the crops, or close to the planted seeds, etc. In another embodiment, multiple environmental sensing devices are buried at different depths respectively.

In step S12, the environmental sensing device can be harvested together with the crops by heavy machinery.

In steps S13 and S14, the environmental data of the processing unit 14 can be reset as the environment changes, so that the sensing result is kept accurate and relevant to different stages of the crops. In the application method of the environmental sensing device according to the present invention, environment conditions may differ at different stages of the crops. For instance, environmental conditions around the crops during transportation are likely to be different to those of the crops during growth. The environmental data can be reset and continuously monitored by the environmental sensing device when the crops changes from one environment to another. Subsequently, environmental conditions such as temperature and humidity, etc., around the crops can then be managed as desired.

The present invention further provides a method for measuring environmental condition around crops, comprising the following steps:

S21. burying the environmental sensing device in the soil where the crops grow when planting the crops. The environmental sensing device may comprise a body, at least one first conducting unit, at least one second conducting unit, an electric measuring unit, a communication unit, a positioning unit, a temperature sensing unit, a processing unit.

The body may comprise an accommodating space inside. The electric measuring unit, the communication unit, the positioning unit, the temperature sensing unit and the processing unit may be disposed in the accommodating space.

The at least one first conducting unit and the at least one second conducting unit may be disposed outside the body and the at least one second conducting unit may correspond to the first conducting unit; wherein the at least one first conducting unit and the at least one second conducting unit may contact the soil where the crops grow.

The electric measuring unit may be for measuring current, voltage, capacitance and temperature, etc., of the surrounding environment corresponding to the first conducting unit and the second conducting unit; wherein the electric measuring unit further transmits a first signal according to the measurement of the current, voltage, capacitance and temperature, etc., of the environment.

The communication unit may be electrically connected to the processing unit for wirelessly communicating with an external data processing device.

The positioning unit may be electrically connected to the processing unit for tracking a location of the environment sensing device.

The temperature sensing unit may be for sensing temperature of the surrounding environment and transmitting a second signal.

The communication unit may transmit a third signal according to the location of the environment sensing device to a database, wherein the database pre-saves worldwide geographic information and the database sends the geographic information of the location of the environment sensing device to the communication unit. According to the geographic information from the database, the processing unit may switch the moisture profile to match the soil type that the environmental sensing device is geographically placed in. This may provide a more accurate moisture reading, since moisture behaves differently in different soil types.

The processing unit may receive the first signal and the second signal and calibrating the measurement of the temperature of the surround environment by the second signal; and the processing unit may calculate soil moisture of the soil where the crops are planted according to the environmental data, the measurement of the current, voltage and capacitance of the surrounding environment, the calibrated temperature of the surrounding environment and the geographic information, etc.

In an embodiment, the method for measuring environmental condition around crops may further comprise the following steps:

S22. harvesting the environmental sensing device together with the crop when harvesting the crops.

In step S22, the method for measuring environmental condition around crops may further include disposing an acceleration sensing unit in the accommodating space and electrically connecting the acceleration sensing unit to the processing unit for sensing an external force received by the body, and measuring g-forces incurred during harvest or transportation by the acceleration sensing unit. According to the present invention, the external forces may be incurred by other object hitting the environmental sensing device or otherwise by the environmental sensing device hitting something else.

In an embodiment, the method for measuring environmental condition around crops may further comprise the following steps:

S23. transporting the environmental sensing device together with the crop when transporting the crops.

In step S23, the method for measuring environmental condition around crops may further disposing a humidity sensing unit in the accommodating space for sensing humidity of the surrounding environment of the crops; and tracking a location of the environment sensing device and the crops by the positioning unit; sensing temperature of the surrounding environment of the crops by the temperature sensing unit; sensing an external force received by the body and measuring g-forces incurred during transportation by the acceleration sensing unit; and transmitting the humidity, the location, the temperature and the external force and the g-force to an external data processing device by the communication unit to allow users to monitor the environmental conditions while the crops are in transit.

In an embodiment, the method for measuring environmental condition around crops may further comprise the following steps:

S24. storing the environmental sensing device together with the crops when the crops are stored.

In step S24, the method for measuring the environmental condition around crops may include sensing humidity of the surrounding environment of the crops by the humidity sensing unit; tracking a location of the environment sensing device and the crops by the positioning unit; sensing temperature of the surrounding environment of the crops by the temperature sensing unit; and transmitting the humidity, the location and the temperature to an external data processing device by the communication unit to allow users to monitor the environmental conditions while in storage.

The environmental sensing device according to the present invention comprises at least one first conducting unit and at least one second conducting unit disposed outside the body. The environmental sensing device measures current, voltage, capacitance and temperature of the surrounding environment corresponding to the first conducting unit and the second conducting unit by the electric measuring unit and calculates a moisture of the surrounding environment through the processing unit. Compared to conventional moisture sensing devices, such as those operate based on osmotic balance, the moisture provided by the environmental sensing device according to the present invention is more accurate and reliable because it is calculated according to the capacitance combined with other environmental data and a unique noise filtering algorithm. Also, the environmental sensing device according to the present invention can provide real-time moisture levels to users which allows the users to precisely irrigate their crop accordingly, so inappropriate actions such as over watering or under watering, etc., can be prevented. Subsequently, utilizing the environmental sensing device according to the present invention to farm can be more economical as the consumed water, fuel or electricity, etc., can be effectively reduced. In addition, users may receive the in-field conditions wirelessly by environmental sensing device according to the present invention and may not need to visit every fields to evaluate the conditions physically, which would save labor, fuel and time, etc. This allows the users to grasp the status of all the farms, crops and/or relative operations, etc, with significant simplicity and efficiency.

Furthermore, the environmental sensing device according to the present invention provides reliability and robustness as the electronic components are substantially placed and covered fully in the accommodating space of the body. In addition, the environmental sensing device according to the present invention is able to continuously monitor the environmental conditions of the crops of different stages of planting, transport and storage, etc. The positioning unit of the environmental sensing device according to the present invention allows users to also track crops throughout the supply chain process, which improves accountability and traceability. In addition, the communication unit or other sensing components can be added, so more information can be collected to obtain environmental conditions with more accuracy, or more functions (e.g. communication) can be added to provide more flexible in applying the environmental sensing device.

The foregoing descriptions of the detailed embodiments are only illustrated to disclose the features and functions of the present invention and not restrictive of the scope of the present invention. It should be understood to those in the art that all modifications and variations according to the spirit and principle in the disclosure of the present invention should fall within the scope of the appended claims.

What is claimed is:

1. An environmental sensing device comprising:
a body having an accommodating space inside;
at least one first conducting unit disposed outside the body;
at least one second conducting unit disposed outside the body and corresponding to the first conducting unit;
an electric measuring unit disposed in the accommodating space for measuring current, voltage, capacitance and temperature of the surrounding environment corresponding to the first conducting unit and the second conducting unit; wherein the electric measuring unit further transmits a first signal according to the measurement of the current, voltage, capacitance and temperature of the environment;
a processing unit disposed in the accommodating space for receiving the first signal and calculating moisture of the surrounding environment according to an environmental data and the measurements of the current, voltage, capacitance and temperature of the surrounding environment; and
a power supply unit disposed in the accommodating space for supplying electric power to the electric measuring unit and the processing unit.

2. The environmental sensing device of claim 1, further comprising:
a temperature sensing unit disposed in the accommodating space for sensing temperature of the surrounding environment and transmitting a second signal;
the processing unit is further used to receive the second signal and calibrating the measurement of the temperature of the surround environment by the second signal; and
wherein the processing unit further calculates the moisture of the surrounding environment according to the environmental data, the measurement of the current, voltage and capacitance of the surrounding environment and the calibrated temperature of the surrounding environment.

3. The environmental sensing device of claim 1, further comprising:
a communication unit disposed in the accommodating space and electrically connected to the processing unit for communicating with an external data processing device.

4. The environmental sensing device of claim 3, further comprising:
a positioning unit disposed in the accommodating space and electrically connected to the processing unit for tracking a location of the environment sensing device.

5. The environmental sensing device of claim 4, further comprising:
a temperature sensing unit disposed in the accommodating space for sensing temperature of the surrounding environment and transmitting a second signal;
the communication unit transmits a third signal according to the location of the environment sensing device to a database, wherein the database pre-saves worldwide geographic information;
the database sends the geographic information of the location of the environment sensing device to the communication unit according to the third signal;
the processing unit is further used to receive the second signal and calibrating the measurement of the temperature of the surround environment by the second signal; and
wherein the processing unit further calculates the moisture of the surrounding environment according to the environmental data, the measurement of the current, voltage and capacitance of the surrounding environment, the calibrated temperature of the surrounding environment and the geographic information.

6. The environmental sensing device of claim 1, further comprising:
a storage unit disposed in the accommodating space for receiving and recording the measurement of the current, voltage, capacitance and temperature of the surrounding environment, the moisture calculated by the processing unit and the corresponding time.

7. The environmental sensing device of claim 1, further comprising:
an acceleration sensing unit disposed in the accommodating space and electrically connected to the processing unit for sensing an external force received by the body.

8. The environmental sensing device of claim 1, further comprising:
a fixing frame disposed in the accommodating space, wherein the fixing frame has at least one cavity for fixing components disposed in the accommodating space.

9. The environmental sensing device of claim 1, wherein the first conducting unit and the second conducting unit are annular and disposed around the outside of the body.

10. The environmental sensing device of claim 1, wherein the first conducting unit and the second conducting unit are made of stainless steel.

11. An application method of the environmental sensing device of claim 1, comprising:
burying the environmental sensing device in the soil in which crops grow when planting the crops;
harvesting the environmental sensing device together with the crop when harvesting the crops;
transporting the environmental sensing device together with the crops when transporting the crops; and
storing the environmental sensing device together with the crop when the crops are stored.

12. A method for measuring environmental condition around crops, comprising:
burying the environmental sensing device in the soil where the crops grow when planting the crops; wherein
the environmental sensing device comprises:
a body having an accommodating space inside;
at least one first conducting unit disposed outside the body;
at least one second conducting unit disposed outside the body and corresponding to the first conducting unit; wherein
the at least one first conducting unit and the at least one second conducting unit touch the soil where the crops grow;
an electric measuring unit disposed in the accommodating space for measuring current, voltage, capacitance and temperature of the surrounding environment corresponding to the first conducting unit and the second conducting unit; wherein the electric measuring unit further transmits a first signal according to the measurement of the current, voltage, capacitance and temperature of the environment;
a communication unit disposed in the accommodating space and electrically connected to the processing unit for communicating with an external data processing device;
a positioning unit disposed in the accommodating space and electrically connected to the processing unit for tracking a location of the environment sensing device;
a temperature sensing unit disposed in the accommodating space for sensing temperature of the surrounding environment and transmitting a second signal;
the communication unit transmits a third signal according to the location of the environment sensing device to a database, wherein the database pre-saves worldwide geographic information;
the database sends the geographic information of the location of the environment sensing device to the communication unit according to the third signal; and
a processing unit disposed in the accommodating space for receiving the first signal and the second signal and calibrating the measurement of the temperature of the surround environment by the second signal; and
wherein the processing unit further calculates soil moisture of the soil where the crops grow according to the environmental data, the measurement of the current, voltage and capacitance of the surrounding environment, the calibrated temperature of the surrounding environment and the geographic information.

13. The method for measuring environmental condition around crops of claim 12, further comprising:
harvesting the environmental sensing device together with the crop when harvesting the crops;
disposing an acceleration sensing unit in the accommodating space and electrically connecting the acceleration sensing unit to the processing unit for sensing an external force received by the body and measuring g-forces incurred during harvest by the acceleration sensing unit; and
transmitting the external force and the g-force to an external data processing device by the communication unit.

14. The method for measuring environmental condition around crops of claim 13, further comprising:
transporting the environmental sensing device together with the crop when transporting the crops;
disposing a humidity sensing unit in the accommodating space for sensing humidity of the surrounding environment of the crops;
tracking a location of the environment sensing device and the crops by the positioning unit;
sensing temperature of the surrounding environment of the crops by the temperature sensing unit; and
transmitting the humidity, the location and the temperature to an external data processing device by the communication unit to allow users to monitor the environmental conditions while in transit.

15. The method for measuring environmental condition around crops of claim 14, further comprising:
storing the environmental sensing device together with the crop when the crops are stored;
sensing humidity of the surrounding environment of the crops by the humidity sensing unit;
tracking a location of the environment sensing device and the crops by the positioning unit;
sensing temperature of the surrounding environment of the crops by the temperature sensing unit; and
transmitting the humidity, the location and the temperature to an external data processing device by the communication unit to allow users to monitor the environmental conditions while in storage.
